(19) United States
(12) Patent Application Publication
(10) Pub. No.: US 2023/0363993 A1
(43) Pub. Date: Nov. 16, 2023

(54) TWO-PHASE COMPOSITION COMPRISING AN AQUEOUS PHASE COMPRISING ONE OR MORE POLYOLS AT A CONTENT RANGING FROM 5% TO 30% BY WEIGHT AND AN OILY PHASE COMPRISING NON-SILICONE OILS

(71) Applicant: L'OREAL, Paris (FR)

(72) Inventors: Caroline SIRICHANDRA, Chevilly La Rue (FR); Christophe DUMOUSSEAUX, Kanagawa (JP); Amélie FRIOT-DARDERES, Chevilly La Rue (FR)

(21) Appl. No.: 17/786,582

(22) PCT Filed: Dec. 15, 2020
(86) PCT No.: PCT/EP2020/086277

§ 371 Date: Jun. 17, 2022

(30) Foreign Application Priority Data
Dec. 20, 2019 (FR) 1915219

(51) Int. Cl. A61K 8/31; A61K 8/03; A61K 8/34; A61K 8/33; A61K 8/92; A61K 8/37; A61Q 19/00

(52) U.S. Cl. CPC A61K 8/31; A61K 8/03; A61K 8/33; A61K 8/345; A61K 8/37; A61K 8/922; A61Q 19/007; A61K 2800/882

(57) ABSTRACT

The present application relates to a two-phase composition comprising an aqueous phase comprising one or more polyols in which the polyol(s) are present in a content ranging from 5% to 30% by weight relative to the total weight of the two-phase composition, and an oily phase comprising one or more non-silicone oils, the oily phase having a viscosity of less than 75 mPa.s according to the viscosity measurement method and the respective weight ratio between the aqueous phase and the oily phase ranging from 60:40 to 95:5..

The present invention relates to a two-phase composition comprising an aqueous phase comprising one or more polyols in a content ranging from 5% to 30% by weight relative to the total weight of the two-phase composition, and an oily phase comprising one or more non-silicone oils and having a viscosity of less than 75 mPa.s according to the viscosity measurement method described herein below and the respective weight ratio between the aqueous phase and the oily phase ranging from 60:40 to 95:5.

A subject of the invention is also a cosmetic method for treating and/or caring for human keratin materials, preferably the skin, comprising the shaking of said two-phase composition and then the application thereof to the surface of said keratin materials.

Another subject of the invention is the use of said composition for the cosmetic treatment of keratin materials, in particular for moisturizing the keratin materials.

### TECHNICAL FIELD

The skin, which is a protective and exchange barrier with the environment, is both strong and fragile, it may lose its suppleness and its capacity to retain water decreases, then causing skin dryness.

It is important to ensure a sufficient level of skin moisturization in order to preserve its suppleness, softness, tonicity and/or appearance. In order to combat the signs of skin dehydration, it is known to apply to the skin cosmetic compositions which promote moisturization in the zones of application. The moisturizing active agents conventionally used, such as humectants, moisturizing polymers, fatty substances such as petroleum jelly or polyols, temporarily modify the superficial properties of the skin by being capable for example of retaining water on the superficial layers of the skin.

However, there is a need to provide novel, in particular moisturizing, compositions exhibiting good cosmetic properties while having good sensory properties such as the absence of a greasy or tacky sensation after application.

Cosmetic compositions can be in the form of a “two-phase composition”, that is to say a composition constituted of two distinct phases, particularly a hydrophilic aqueous phase and an oily phase. They differ from emulsions in that, when at rest, the two phases are separate instead of being emulsified one in the other. Thus, the two phases are separated at rest by a single interface, whereas, for emulsions, one of the phases is dispersed in the other in the form of a multitude of droplets, and the interfaces are therefore multiple.

The use of two-phase compositions necessitates prior shaking in order to form an extemporaneous emulsion. This emulsion must be of sufficient quality and stability to enable homogeneous application of two phases, but at rest, the two phases must become rapidly separated and regain their initial state, this phenomenon being known as “phase separation”. After mixing, the interface between the two phases must rapidly become clearly defined in order to guarantee the esthetics of the product.

Furthermore, an increasing number of consumers are looking for natural and/or naturally derived cosmetic products, in particular products which do not comprise any silicone compounds, while at the same time remaining very demanding with regard to the sensory qualities and to the performance quality thereof.

Two-phase compositions have already been described, for example in the patent application FR2939662. Such compositions may make it possible to obtain, after shaking, a transparent emulsion, while retaining the properties of the two-phase compositions as regards rapid phase separation into two transparent phases. However, these compositions can exhibit significant concentrations of polyols, resulting in a tacky sensation and a sensation of greasy skin after application.

There thus remains the need to provide cosmetic, in particular moisturizing, compositions in the form of two-phase compositions that are devoid of the above-mentioned drawbacks.

Thus, the aim of the present invention is to develop a cosmetic, in particular moisturizing, composition in the form of a two-phase composition having good cosmetic properties such as a good moisturizing efficacy, good sensory properties such as the absence of tackiness after application of the composition, while having a clearly defined interface between the two phases and a phase separation time which is suitable for allowing the use of the product, and also a rapid return to two distinct phases.

### DISCLOSURE OF THE INVENTION

One subject of the present invention is a two-phase composition comprising:
a) an aqueous phase comprising one or more polyols in which the polyol(s) are present in a content ranging from 5% to 30% by weight relative to the total weight of the two-phase composition, and
b) an oily phase comprising one or more non-silicone oils,
the oily phase having a viscosity of less than 75 mPa.s according to the viscosity measurement method and the respective weight ratio between the aqueous phase and the oily phase ranging from 60:40 to 95:5.

The present invention also relates to a cosmetic method for treating and/or caring for human keratin materials, preferably the skin, comprising the shaking of a two-phase composition as defined hereinabove and then the application thereof to the surface of said keratin materials.

The present invention also relates to a kit comprising:
i) a composition comprising an aqueous phase comprising one or more polyols in which the polyol(s) are present in a content ranging from 5% to 30% by weight relative to the total weight of the two-phase composition; and
ii) a composition comprising an oily phase comprising one or more non-silicone oils, the oily phase having a viscosity of less than 75 mPa.s according to the viscosity measurement method and the respective weight ratio between the aqueous phase and the oily phase ranging from 60:40 to 95:5.

The compositions i) and ii) of the kit according to the invention are preferably identical to the aqueous a) and oily b) phases of the composition, respectively.

A subject of the present invention is also a cosmetic method for treating and/or caring for human keratin materials, preferably the skin, in particular for moisturizing the keratin materials, comprising the preparation of a two-phase composition by mixing and shaking a composition i) with a composition ii) of the kit according to the invention, and then applying this to the surface of said keratin materials.

It has thus been demonstrated that a two-phase composition, which comprises an aqueous phase having a polyol content lower than the contents described in the prior art and also an oily phase with a certain viscosity, had good sensory properties, in particular of freshness on application, an absence of tackiness and of greasy sensation after application, while having a good skin moisturizing sensation after application.

In addition, after shaking the two-phase composition according to the invention, the two phases, i.e. aqueous phase and oily phase, separate rapidly to return to their initial state; the interface between the two phases is clearly defined, which guarantees the esthetics of the product.

Lastly, with the two-phase composition according to the invention, the phase separation time is suitable for allowing both the use of the product by the consumer and a rapid return to two distinct phases.

The invention is not limited to the illustrated examples. The features of the various examples may notably be combined within variants which are not illustrated.

The term “at least one” means one or more.

The term two-phase composition or “biphasic composition” denotes a composition which is constituted of two distinct phases, in this case an aqueous phase and an oily phase, and which requires prior shaking in order to form an emulsion before application.

According to the invention, the term “keratin materials” means the skin of the body, face and/or area around the eyes, the lips, the nails, the mucous membranes, the eyelashes, the eyebrows, bodily hair, the scalp and/or the hair, or any other area of bodily skin. More particularly, the keratin materials according to the invention are the scalp, the hair and/or the skin.

Preferably, the keratin materials according to the invention are the skin.

The term “skin” means all of the skin of the body, and preferably the skin of the face, neckline, neck, arms and forearms, or even more preferably the skin of the face, in particular of the forehead, nose, cheeks, chin and area around the eyes.

All of the compositions according to the present invention are cosmetic compositions. The term “cosmetic” means a composition that is compatible with the skin, the mucous membranes and the integuments.

Preferably, the two-phase composition according to the invention is a cosmetic composition, more preferentially a cosmetic composition for the treatment of keratin materials such as the skin.

### Aqueous Phase

The composition according to the invention comprises at least one aqueous phase. The term “aqueous phase” means a phase comprising water and generally any molecule in dissolved form in the water in the composition. The aqueous phase is preferably an aqueous solution.

The aqueous phase according to the invention comprises water. By preference, the water is present at a content of at least 50% by weight relative to the total weight of the aqueous phase, preferably at least 60% by weight, more preferentially at least 65% by weight and better still at least 70% by weight, relative to the total weight of the aqueous phase.

The composition according to the invention may comprise water in a content of greater than or equal to 30% by weight relative to the total weight of the two-phase composition.

By preference, the composition comprises water in a total amount of between 30% and 90% by weight, better still between 40% and 85% by weight and even better still between 50% and 80% by weight, relative to the total weight of the two-phase composition.

The concentration of the aqueous phase by preference varies from 60% to 95% by weight, preferably from 70% to 95% by weight and more preferentially from 75% to 90% by weight, relative to the total weight of the two-phase composition.

### Polyol

The aqueous phase comprises one or more polyols.

For the purposes of the present invention, the term “polyol” means an organic compound constituted of a hydrocarbon-based chain optionally interrupted with one or more oxygen atoms and bearing at least two free hydroxyl groups (—OH) borne by different carbon atoms, this compound possibly being cyclic or acyclic, linear or branched, and saturated or unsaturated.

More particularly, the polyol(s) comprise from 2 to 30 hydroxyl groups, more preferentially from 2 to 10 hydroxyl groups and even more preferentially from 2 to 3 hydroxyl groups.

The polyol(s) are preferably chosen from glycerol, propylene glycol, propane-1,3-diol, 1,3-butylene glycol, pentane-1,2-diol, dipropylene glycol, hexylene glycol, ethylene glycol, polyethylene glycols, sorbitol, sugars such as glucose and mixtures thereof.

The polyol(s) are more preferentially chosen from glycerol, propane-1,3-diol, pentylene glycol, 1,3-butylene glycol and mixtures thereof, better still glycerol, propane-1,3-diol, and mixtures thereof.

According to a preferred embodiment, the aqueous phase comprises a mixture of glycerol and propane-1,3-diol.

Preferably, the aqueous phase comprises one or more polyols in a content ranging from 5% to 30% by weight, more preferentially ranging from 5% to 25% by weight, better still from 10% to 20% by weight, relative to the total weight of the aqueous phase.

The composition according to the invention comprises one or more polyols in a content ranging from 5% to 30% by weight, more preferentially ranging from 5% to 25% by weight, better still from 10% to 20% by weight, relative to the total weight of the two-phase composition.

The aqueous phase may also comprise a primary alcohol, that is to say an alcohol comprising from 1 to 6 carbon atoms, such as ethanol and isopropanol. Preferably, the aqueous phase comprises ethanol.

The primary alcohol(s) may be present in an amount ranging from 0.01% to 15% by weight, and preferably from 0.1% to 10% by weight, relative to the total weight of the aqueous phase.

### Moisturizing Active Agent

The aqueous phase may comprise one or more moisturizing active agents chosen from urea, hydroxyethylurea, hyaluronic acid, glycine, β-alanine, taurine, trimethylglycine, and mixtures thereof.

Trimethylglycine, also called “glycine betaine” or “betaine”, is a quaternary amino acid. At neutral pH, the compound exists in the form of a zwitterionic compound, forming a salt between the quaternary ammonium function and the carboxyl function of the molecule. In the presence of strong acids, it will form acid addition salts, for example hydrochloride. The compound was originally isolated from sugar beet.

Preferably, the moisturizing active agent is trimethylglycine.

Preferably, the composition comprises trimethylglycine as moisturizing active agent.

Mention may be made, by way of example, of the trimethylglycine sold by the company Danisco under the trade name GENENCARE OSMS BA (INCI name: Betaine).

The aqueous phase according to the invention can comprise one or more moisturizing active agents present in amounts ranging from 0.1% to 10% by weight, preferably from 0.5% to 6% by weight and more preferentially from 0.5% to 5% by weight, relative to the total weight of the aqueous phase.

The two-phase composition according to the invention can comprise one or more moisturizing active agents present in amounts ranging from 0.1% to 10% by weight, preferably from 0.5% to 6% by weight and more preferentially from 0.5% to 5% by weight, relative to the total weight of the two-phase composition.

### Oily Phase

The composition according to the invention comprises at least one oily phase.

The oily phase generally comprises one or more hydrophobic compounds which render said phase water-immiscible. Said phase is liquid (in the absence of structuring agent) at room temperature (20-25° C.),

The concentration of the oily phase preferably varies from 5% to 40% by weight, better still from 5% to 30% by weight and even better still from 10% to 25% by weight, relative to the total weight of the two-phase composition.

The oily phase has a viscosity of less than 75 mPa.s, measured in accordance with the viscosity measurement method as described in example 3.

The oily phase preferably has a viscosity of less than 70 mPa.s, more preferentially of less than 60 mPa.s, according to the viscosity measurement method described hereinbelow.

Having an oily phase with a viscosity of less than 75 mPa.s makes it possible to obtain a good quality of interface between the aqueous phase and the oily phase and in particular a clearly defined interface.

### Non-Silicone Oils

The oily phase comprises one or more non-silicone oils.

Preferably, the oily phase comprises, as non-silicone oils, one or more volatile or non-volatile hydrocarbon-based oils.

The term “oil” means a fatty substance that is liquid at room temperature (25° C.) and atmospheric pressure (760 mmHg, i.e. 10⁵ Pa). The oil may be volatile or non-volatile.

For the purposes of the invention, the term “volatile oil” means an oil that is capable of evaporating on contact with the skin or the keratin fiber in less than one hour, at room temperature and atmospheric pressure. The volatile oils of the invention are volatile cosmetic oils, which are liquid at room temperature, having a non-zero vapor pressure, at room temperature and atmospheric pressure, ranging in particular from 0.13 Pa to 40,000 Pa (10⁻³ to 300 mmHg), in particular ranging from 1.3 Pa to 13,000 Pa (0.01 to 100 mmHg) and more particularly ranging from 1.3 Pa to 1300 Pa (0.01 to 10 mmHg).

The term “non-volatile oil” means an oil that remains on the skin or the keratin fiber at room temperature and atmospheric pressure for at least several hours, and that notably has a vapor pressure of less than 10⁻³ mmHg (0.13 Pa).

The term “hydrocarbon-based oil” means an oil mainly containing carbon and hydrogen atoms and possibly one or more functions chosen from hydroxyl, ester, ether and carboxylic functions.

As examples of volatile hydrocarbon-based oils that may be used in the invention, mention may be made of:
volatile hydrocarbon-based oils containing from 8 to 16 carbon atoms, and especially C₈-C₁₆ isoalkanes of petroleum origin (also known as isoparaffins), for instance isododecane (also known as 2,2,4,4,6-pentamethylheptane), isodecane and isohexadecane, for example the oils sold under the trade names Isopar or Permethyl, branched C₈-C₁₅ esters and isohexyl neopentanoate, and mixtures thereof. Other volatile hydrocarbon-based oils, for instance petroleum distillates, especially those sold under the name Shell Solt by the company Shell, may also be used;
volatile linear alkanes comprising from 7 to 26 carbon atoms, such as undecane, tridecane. As examples of linear alkanes that are suitable for use in the invention, mention may be made of n-heptane (C7), n-octane (C8), n-nonane (C9), n-decane (C10), n-undecane (C11), n-dodecane (C12), n-tridecane (C13) and n-tetradecane (C14), and mixtures thereof.

As examples of non-volatile hydrocarbon-based oils that may be used in the invention, mention may be made of:
hydrocarbon-based plant oils such as liquid triglycerides of fatty acids of 4 to 24 carbon atoms, for instance heptanoic or octanoic acid triglycerides, or wheatgerm oil, olive oil, sweet almond oil, palm oil, rapeseed oil, coconut oil, cottonseed oil, alfalfa oil, poppy oil, pumpkin oil, marrow oil, blackcurrant oil, evening primrose oil, millet oil, barley oil, quinoa oil, rye oil, safflower oil, candlenut oil, passion flower oil, musk rose oil, sunflower oil, corn oil, soybean oil, marrow oil, grapeseed oil, sesame seed oil, hazelnut oil, apricot kernel oil, macadamia oil, castor oil, avocado oil, jojoba oil, shea butter or caprylic/capric acid triglycerides, for instance those sold by the company Stéarinerie Dubois or those sold under the names Miglyol 810, 812 and 818 by the company Dynamit Nobel;
synthetic ethers containing from 10 to 40 carbon atoms, such as dimethyl ether;
synthetic esters, especially of fatty acids, for instance the oils of formula R1COOR2 in which R1 represents a linear or branched higher fatty acid residue containing from 1 to 40 carbon atoms and R2 represents a hydrocarbon-based chain, which is especially branched, containing from 1 to 40 carbon atoms, with R1 + R2 ≥ 10, for instance PurCellin oil (cetostearyl octanoate), isononyl isononanoate, isopropyl myristate, isopropyl palmitate, C12 to C15 alcohol benzoate, hexyl laurate, diisopropyl adipate, isononyl isononanoate, 2-ethylhexyl palmitate, 2-octyldodecyl stearate, 2-octyldodecyl erucate, isostearyl isostearate or tridecyl trimellitate; alcohol or polyalcohol octanoates, decanoates or ricinoleates, for instance propylene glycol dioctanoate; hydroxylated esters, for instance isostearyl lactate, octyl hydroxystearate, octyldodecyl hydroxystearate, triisocetyl citrate, and fatty alcohol heptanoates, octanoates or decanoates; polyol esters, for instance propylene glycol dioctanoate, neopentyl glycol diheptanoate or diethylene glycol diisononanoate; and pentaerythritol esters, for instance pentaerythrityl tetraisostearate; according to a preferred embodiment of the invention, the oily phase does not comprise diisostearyl malate;
linear or branched hydrocarbons, of mineral or synthetic origin, such as liquid paraffins and derivatives thereof, petroleum jelly, polydecenes, polybutenes, hydrogenated polyisobutene such as Parleam, or squalane;
fatty alcohols which are liquid at room temperature and have a branched and/or unsaturated carbon chain containing from 12 to 26 carbon atoms, such as octyldodecanol, isostearyl alcohol, 2-butyloctanol, 2-hexyldecanol, 2-undecylpentadecanol or oleyl alcohol;
higher fatty acids, such as oleic acid, linoleic acid or linolenic acid;
carbonates;
acetates; or
citrates.

More preferentially, the non-silicone oil(s) are chosen from volatile linear alkanes comprising from 7 to 26 carbon atoms, hydrocarbon-based plant oils such as apricot kernel oil or caprylic/capric acid triglycerides; linear or branched hydrocarbons of mineral or synthetic origin, such as Parleam or squalane; fatty alcohols which are liquid at room temperature and have a branched and/or unsaturated carbon chain containing from 12 to 26 carbon atoms, such as octyldodecanol; and mixtures thereof.

By preference, the non-silicone oil(s) are present in a content of at least 85% by weight relative to the total weight of oily phase, preferably at least 90% by weight and preferably at least 95% by weight, relative to the total weight of the oily phase.

The two-phase composition according to the invention can comprise one or more non-silicone oils in a content ranging from 5% to 40% by weight, better still from 7% to 30% by weight and even better still from 9% to 25% by weight, relative to the total weight of the two-phase composition.

The composition according to the invention can comprise less than 2% by weight of silicone oils relative to the total weight of the composition, preferably less than 1% by weight of silicone oils relative to the total weight of the composition. More preferentially, the two-phase composition is free of silicone oils.

The term “silicone oil” means an oil containing at least one silicon atom, and in particular containing Si—O groups.

Examples of silicone oils that may be mentioned include volatile silicone oils such as cyclopolydimethylsiloxanes (INCI name: cyclomethicone), such as cyclopentasiloxane, cyclohexasiloxane, octamethylcyclotetrasiloxane, decamethylcyclopentasiloxane, dodecamethylcyclohexasiloxane; linear silicones such as heptamethylhexyltrisiloxane, heptamethyloctyltrisiloxane, hexamethyldisiloxane, octamethyltrisiloxane, decamethyltetrasiloxane or dodecamethylpentasiloxane; non-volatile silicone oils such as polymethylsiloxanes (PDMS), and phenyl polymethylsiloxanes such as phenyl trimethicones, phenyl dimethicones, phenyltrimethylsiloxydiphenylsiloxanes, diphenyl dimethicones, diphenylmethyldiphenyltrisiloxanes, 2-phenylethyl trimethylsiloxysilicates and polymethylphenylsiloxanes; polysiloxanes modified with fatty acids, fatty alcohols or polyoxyalkylenes, and mixtures thereof.

### Additives

The composition according to the invention, i.e aqueous phase and oily phase, may in addition also comprise additional cosmetic and dermatological active agents.

The composition according to the invention, i.e. aqueous phase and oily phase, may additionally comprise cosmetic adjuvants which will be located in either phase depending on their hydrophilic or lipophilic nature, such as opacifiers, preservatives, sunscreens, fillers, suspension agents, sequestrants, fragrances, colorants or any other ingredient commonly used in cosmetics for this type of application.

Preferably, the two-phase composition according to the invention does not comprise any surfactant.

The two-phase composition according to the invention comprises the aqueous phase and the oily phase in a respective weight ratio ranging from 60:40 to 95:5, preferably ranging from 75:25 to 90:10, preferably of around 90:10.

In a preferred embodiment of the invention, the two-phase composition according to the invention has a pH of between 5 and 7, preferably a pH of between 5 and 6.7.

The two-phase composition according to the invention is liquid.

The term “liquid” means that the composition is sufficiently fluid for it to be shaken and/or for it to be able to be sprayed.

The two-phase composition according to the invention may be contained in a device enabling application thereof, in particular by spraying, onto the skin. This device is preferably a pump bottle.

Preferably, the composition according to the invention comprises a “physiologically acceptable medium”. The term “physiologically acceptable medium” means a medium that is suitable for the topical administration of a composition, and that is compatible with all human keratin materials, such as the skin, the lips, the nails, the mucous membranes, the eyelashes, the eyebrows, the scalp and/or the hair, or any other area of bodily skin.

According to the invention, a physiologically acceptable medium is preferentially a cosmetically acceptable medium, i.e. a medium which is free of any odor or unpleasant appearance and which is entirely compatible with the topical administration route.

More particularly, the composition according to the invention is suitable for topical administration, that is to say for application to the surface of the keratin material under consideration, such as the skin under consideration.

These compositions are in particular intended for topical application to the face and/or the body.

The present invention also relates to the use of a two-phase composition as defined hereinabove for the cosmetic treatment of keratin materials, in particular for moisturizing the keratin materials, preferably the skin,

The present invention also relates to a cosmetic method for treating and/or caring for human keratin materials, preferably the skin, comprising the shaking of a two-phase composition as defined hereinabove and then the application thereof to the surface of said keratin materials. Shaking is typically performed by hand.

The method for treating and/or caring for human keratin materials is preferably a method for moisturizing the keratin materials, in particular the skin.

The present invention also relates to a kit comprising:
i) a composition comprising an aqueous phase comprising one or more polyols in which the polyol(s) are present in a content of between 5% and 30% by weight relative to the total weight of the two-phase composition; and
ii) a composition comprising an oily phase comprising one or more non-silicone oils, the oily phase having a viscosity of less than 75 mPa.s according to the viscosity measurement method and the respective weight ratio between the aqueous phase and the oily phase ranging from 60:40 to 95:5.

The compositions i) and ii) of the kit according to the invention are preferably identical to the aqueous a) and oily b) phases of the composition as described above, respectively. Typically, each composition i) and ii) is liquid.

A subject of the present invention is also a cosmetic method for treating and/or caring for human keratin materials, preferably the skin, in particular for moisturizing the keratin materials, comprising the preparation of a two-phase composition by mixing and shaking a composition i) with a composition ii) of the kit according to the invention, and then applying this to the surface of said keratin materials.

Needless to say, those skilled in the art will take care to select this or these optional additional compounds such that the advantageous properties intrinsically associated with the cosmetic composition in accordance with the invention are not, or are not substantially, adversely affected by the envisaged addition(s).

The following examples serve to illustrate the present invention. The amounts are given as weight percentages relative to the total weight of the composition.

### EXAMPLES

In the examples, the temperature is given in degrees Celsius and corresponds to room temperature (20-25° C.), unless otherwise indicated, and the pressure is atmospheric pressure at sea level, unless otherwise indicated. Moreover, unless otherwise indicated, the percentages are expressed on a weight basis relative to the total weight of the composition.

### Example 1

The following compositions were prepared according to the process below:
phase A (i.e. aqueous phase) is mixed using a stirrer (for example a Turbotest Rayneri from VMI) at a speed of 200-350 rpm and at room temperature, in order to obtain a homogeneous mixture; the mixture is then filtered using a 5 µm nylon filter;
phase B (i.e. oily phase) is then prepared, and
96 g of phase A is weighed into a beaker and to this is added 24 g of phase B, to obtain a two-phase composition.

TABLE 1

| Phase | Chemical name (INCl) | Composition A (invention) | Composition B (invention) | Composition C (invention) | Composition D (invention) | Composition E (comparative) |
|---|---|---|---|---|---|---|
| A | Ethanol | 3 | 3 | 3 | 3 | 3 |
| | Glycerol | 5 | 10 | 20 | 30 | 0 |
| | Preservatives | 1 | 1 | 1 | 1 | 1 |
| | pH adjuster | qs pH = 5 | qs pH = 5 | qs pH = 5 | qs pH = 5 | qs pH = 5 |
| | Chelating agent EDTA | 0.2 | 0.2 | 0.2 | 0.2 | 0.2 |
| | Water | qs 100 | qs 100 | qs 100 | qs 100 | qs 100 |
| B | Squalane | 20 | 20 | 20 | 20 | 20 |

The quality of the interface was evaluated for each composition in accordance with the method described hereinbelow.

### Evaluation of the Quality of the Interface:

For each composition A to E, two 140 ml plastic bottles with 120 g of two-phase formula (96 g of aqueous phase and 24 g of oily phase) are prepared for the evaluation. One of the bottles will be shaken and the other will serve as a reference for the grading.

The evaluation is performed over 5 days:
on the first day (D1), the sample comprising the two-phase composition is shaken back and forth 10 times;
on the second day (D2), the sample is shaken again (back and forth 10 times);
on the third day (D3), the sample is shaken again (back and forth 10 times);
on the fourth day (D4), the sample is shaken again (back and forth 10 times);
on the fifth day (D5), the sample is evaluated by giving a grade for the quality of the interface.

The grades are given on a scale from 1 to 5. Positive (5/5), intermediate (⅗) and negative (⅕) boundaries were formulated and captured by photograph in order to assist in the grading.

A two-phase composition having a grade equal to ⅕ exhibits an interface between the aqueous phase and the oily phase which is in the form of a white foam over the entire height of the oily phase.

A two-phase composition having a grade equal to ⅗ exhibits a milky interface in the form of a fine layer between the aqueous phase and the oily phase.

A two-phase composition having a grade equal to 5/5 exhibits an interface between the aqueous phase and the oily phase which is identical to that of the unshaken reference sample.

Compositions having a grade of less than or equal to 3 for the quality of the interface are considered to be unacceptable from an esthetics point of view.

Each composition A to E received a grade for the quality of the interface.

### Results:

TABLE 2

| | | | | | |
|---|---|---|---|---|---|
| | Composition A | Composition B | Composition C | Composition D | Composition E |
| Interface grade | 3.5 | 3.5 | 3.5 | 4 | 3 |
| Macroscopic observation of the interface | Clearly defined interface | Clearly defined interface | Clearly defined interface | Clearly defined interface | Milky interface |

Comparative composition E has a grade equal to 3 for the interface quality, which is unacceptable from an esthetics point of view.

Compositions A to D according to the invention have grades of strictly greater than 3 for the interface quality. The presence of polyols such as glycerol in the aqueous phase at a concentration ranging from 5% to 30% by weight relative to the total weight of the two-phase composition therefore makes it possible to obtain two-phase compositions with a clearly defined interface between the two phases.

### Example 2

The following compositions were prepared according to the process described in example 1.

TABLE 3

| Phase | Chemical name (INCl) | Composition F (invention) | Composition G (invention) | Composition H (invention) | Composition I (comparative) |
|---|---|---|---|---|---|
| | Ethanol | 3 | 3 | 3 | 3 |
| A | Glycerol | 5 | 10 | 20 | 40 |
| | Preservatives | 1 | 1 | 1 | 1 |
| | pH adjuster | qs pH = 5 | qs pH = 5 | qs pH = 5 | qs pH = 5 |
| | Chelating agent EDTA | 0.2 | 0.2 | 0.2 | 0.2 |
| | Water | qs 100 | qs 100 | qs 100 | qs 100 |
| B | Dicaprylyl ether | 20 | 20 | 20 | 20 |

TABLE 4

| Phase | Chemical name (INCl) | Composition J (invention) | Composition K (invention) | Composition L (invention) | Composition M (comparative) |
|---|---|---|---|---|---|
| A | Ethanol | 3 | 3 | 3 | 3 |
| | Glycerol | 10 | 20 | 30 | 40 |
| | Preservatives | 1 | 1 | 1 | 1 |
| | pH adjuster | qs pH = 5 | qs pH = 5 | qs pH = 5 | qs pH = 5 |
| | Chelating agent EDTA | 0.2 | 0.2 | 0.2 | 0.2 |
| | Water | qs 100 | qs 100 | qs 100 | qs 100 |
| B | Octyldodecan ol | 20 | 20 | 20 | 20 |

The demixing time was evaluated for each composition F to M in accordance with the process described hereinbelow.

### Measurement of the Demixing Time for the Two-Phase Compositions:

Samples are taken of the two-phase compositions to be analyzed (120 g of two-phase formula with 96 g of aqueous phase and 24 g of oily phase) and placed into 140 ml plastic bottles. The samples are then placed in front of a black background in a Basic Color Viewing Light light box from JUST-Normlicht with supermarket-type lighting.

A video camera (Nikon Coolpix P100) is placed in front of the samples comprising the compositions, at approximately 1 m from each sample.

The camera is started and then the samples are shaken back and forth 10 times. The recording continues until the aqueous phase and the oily phase of each two-phase composition separate and regain their initial state. Recording is stopped after 5 minutes even if the two phases of the two-phase compositions have not returned to their initial state.

The two-phase compositions which have a demixing time of greater than or equal to 3 minutes 50 are considered to be unacceptable, because the time for separation of the aqueous phase and the oily phase is not rapid enough and is therefore considered to be unattractive by consumers.

### Results:

TABLE 5

| | Comp. F | Comp. G | Comp. H | Comp. I | Comp. J | Comp. K | Comp. L | Comp. M |
|---|---|---|---|---|---|---|---|---|
| Demixing time (in minutes) | 2.00 | 2.50 | 3.27 | >5 | 2.10 | 2.00 | 2.53 | 3.50 |

Compositions F to H according to the invention and also compositions J, K and L according to the invention have a demixing time of strictly less than 3 minutes 50. These two-phase compositions according to the invention, which comprise a polyol content of between 5% and 30%, have an aqueous phase and an oily phase which separate rapidly to return to their initial state.

The comparative compositions I and M, which comprise a polyol content of strictly greater than 30%, have a demixing time of greater than or equal to 3 minutes 50, which is unacceptable from an esthetics point of view.

### Example 3

The following compositions were prepared according to the process described in example 1.

TABLE 6

| Phase | Chemical name (INCI) | Composition N (comparative) | Composition O (invention) | Composition P (invention) | Composition Q (invention) |
|---|---|---|---|---|---|
| A | Ethanol | 3 | 3 | 3 | 3 |
| | Glycerol | 5 | 5 | 5 | 5 |
| | Preservatives | 1 | 1 | 1 | 1 |
| | pH adjuster | qs pH = 5 | qs pH = 5 | qs pH = 5 | qs pH = 5 |
| | Chelating agent EDTA | 0.2 | 0.2 | 0.2 | 0.2 |
| | Water | qs 100 | qs 100 | qs 100 | qs 100 |
| B | Diisostearyl malate | 20 | - | - | - |
| | Prunus armenincn (apricot) kernel oil | - | 20 | - | - |
| | Squalane | - | - | 20 | - |
| | Caprylic/ capric triglyceride | - | - | - | 20 |
| Viscosity of oily phase (in mPa.s) | | 2091.60 | 55.75 | 28.14 | 22.48 |

TABLE 7

| Phase | Chemical name (INCI) | Composition R (invention) | Composition S (invention) | Composition T (invention) | Composition U (invention) |
|---|---|---|---|---|---|
| A | Ethanol | 3 | 3 | 3 | 3 |
| | Glycerol | 5 | 5 | 5 | 5 |
| | Preservatives | 1 | 1 | 1 | 1 |
| | pH adjuster | qs pH = 5 | qs pH = 5 | qs pH = 5 | qs pH = 5 |
| | Chelating agent EDTA | 0.2 | 0.2 | 0.2 | 0.2 |
| | Water | qs 100 | qs 100 | qs 100 | qs 100 |
| B | Hydrogenated polyisobutene (trade name Parleam®, sold by the company NOF Corporation) | 20 | - | - | - |
| | Dicaprylyl ether | - | 20 | - | - |
| | Octyldode-c anol | - | - | 20 | |
| | C₁₃-C₁₅ alkane (trade name Neossance Hemi -squalane, sold by the company Amyris) | - | - | - | 20 |
| Viscosity of oily phase (in mPa.s) | | 31.64 | 3.21 | 44.66 | 2.40 |

The viscosities of the oily phases of the different compositions are measured according to the process described hereinbelow.

The viscosities of the oily phases are measured using a Stabinger SVM 3001 capillary viscometer from Anton Paar. The measurements are performed at 25° C.

5 ml of each oil are introduced into the viscometer using a syringe. Two or three measurements are performed for each sample according to the reproducibility of the results.

The machine performs a double rinse (with toluene and then with acetone) between each sample. In the end, a mean dynamic viscosity (in mPa.s) is recorded for each oil.

The quality of the interface was evaluated for each composition in accordance with the method described in example 1.

### Results:

TABLE 8

| | Comp. N | Comp. O | Comp. P | Comp. Q | Comp. R | Comp. S | Comp. T | Comp. U |
|---|---|---|---|---|---|---|---|---|
| Interface grade | 1 | 3.3 | 3.5 | 3.5 | 3.8 | 4.5 | 4.7 | 4.7 |

Compositions O to U according to the invention have grades of strictly greater than 3 for the interface quality. The presence of an oily phase having a viscosity of less than 75 mPa.s thus makes it possible to obtain two-phase compositions with a clearly defined interface between the two phases.

Comparative composition N has a grade of less than 3 for the interface quality, which is unacceptable from an esthetics point of view.

### Example 4: Example of a Moisturizing Care Composition

The following compositions were prepared according to the process described in example 1.

TABLE 9

| Phase | Chemical name (INCI) | Composition V (invention) | Composition W (invention) |
|---|---|---|---|
| A | Glycerol | 10 | 10 |
| | Propanediol | 5 | 5 |
| | Betaine | 2 | 2 |
| | Preservatives | qs | qs |
| | pH adjuster | qs pH = 5 | qs pH = 5 |
| | Salicylic acid | 0.2 | 0.2 |
| | Sodium chloride | 0.50 | 0.50 |
| | Water | qs 100 | qs 100 |
| B | Caprylic/capric triglyceride | 10.95 | - |
| | Fragrance | 0.05 | - |
| | Squalane | - | 11.00 |

Compositions V and W according to the invention are mixed and applied to the skin using a pump bottle.

Compositions V and W according to the invention confer a good skin moisturizing sensation with an absence of tackiness and greasy sensation during application.

1. A two-phase composition comprising:
a) an aqueous phase comprising one or more polyols in which the polyol(s) are present in a content ranging from 5% to 30% by weight relative to the total weight of the two-phase composition, and
b) an oily phase comprising one or more non-silicone oils,
the oily phase having a viscosity of less than 75 mPa.s according to the viscosity measurement method; and
the respective weight ratio between the aqueous phase and the oily phase ranging from 60:40 to 95:5.

2. The composition as claimed in claim 1, characterized in that the polyol(s) are chosen from glycerol, propylene glycol, propane-1,3-diol, 1,3-butylene glycol, pentane-1,2-diol, dipropylene glycol, hexylene glycol, ethylene glycol, polyethylene glycols, sorbitol, sugars and mixtures thereof.

3. The composition as claimed in claim 1, characterized in that the polyol(s) are chosen from glycerol, propane-1,3-diol, pentylene glycol, 1,3-butylene glycol and mixtures thereof, better still glycerol, propane-1,3-diol, and mixtures thereof.

4. The composition as claimed in claim 1, characterized in that the aqueous phase comprises one or more polyols in a content ranging from 5% to 25% by weight relative to the total weight of the two-phase composition.

5. The composition as claimed in claim 1, characterized in that the aqueous phase comprises one or more moisturizing active agents chosen from urea, hydroxyethylurea, hyaluronic acid, glycine, β-alanine, taurine, trimethylglycine, and mixtures thereof.

6. The composition as claimed in claim 5, characterized in that the aqueous phase comprises one or more moisturizing active agents present in amounts ranging from 0.1% to 10% by weight relative to the total weight of the two-phase composition.

7. The composition as claimed in claim 1, characterized in that the oily phase comprises, as non-silicone oils, one or more volatile or non-volatile hydrocarbon-based oils.

8. The composition as claimed in claim 1, characterized in that the non-silicone oil(s) are chosen from volatile linear alkanes comprising from 7 to 26 carbon atoms, hydrocarbon-based plant oils; linear or branched hydrocarbons of mineral or synthetic origin; fatty alcohols which are liquid at room temperature and have a branched and/or unsaturated carbon chain containing from 12 to 26 carbon atoms; and mixtures thereof.

9. The composition as claimed in claim 1, characterized in that it comprises one or more non-silicone oils in a content ranging from 5% to 40% by weight relative to the total weight of the two-phase composition.

10. The composition as claimed in claim 1, characterized in that the oily phase has a viscosity of less than 70 mPa.s according to the viscosity measurement method.

11. The composition as claimed in claim 1, characterized in that it comprises the aqueous phase and the oily phase in a respective weight ratio ranging from 75:25 to 90:10.

12. A cosmetic method for treating and/or caring for human keratin materials, comprising the shaking of a two-phase composition as claimed in claim 1 and then the application thereof to the surface of said keratin materials.

13. The method as defined in claim 12 for for moisturizing the keratin materials.

14. A kit comprising:
i) a composition comprising an aqueous phase comprising one or more polyols in which the polyol(s) are present in a content ranging from 5% to 30% by weight relative to the total weight of the two-phase composition; and
ii) a composition comprising an oily phase comprising one or more non-silicone oils, the oily phase having a viscosity of less than 75 mPa.s according to the viscosity measurement method; and the respective weight ratio between the aqueous phase and the oily phase ranging from 60:40 to 95:5.

15. A cosmetic method for treating and/or caring for human keratin materials comprising the preparation of a two-phase composition by mixing and shaking a composition i) with a composition ii) of the kit as claimed in claim 14, and then applying this to the surface of said keratin materials.

16. The composition as claimed in_claim 1, characterized in that the polyol(s) are chosen from glycerol, propane-1,3-diol, and mixtures thereof.

17. The composition as claimed in claim 2, characterized in that the aqueous phase comprises one or more polyols in a content ranging from 5% to 25% by weight relative to the total weight of the two-phase composition.

18. The composition as claimed in claim 3, characterized in that the aqueous phase comprises one or more polyols in a content ranging from 5% to 25% by weight relative to the total weight of the two-phase composition.

19. The composition as claimed in claim 2, characterized in that the aqueous phase comprises one or more moisturizing active agents chosen from urea, hydroxyethylurea, hyaluronic acid, glycine, β-alanine, taurine, trimethylglycine, and mixtures thereof.

20. The composition as claimed in claim 3, characterized in that the aqueous phase comprises one or more moisturizing active agents chosen from urea, hydroxyethylurea, hyaluronic acid, glycine, β-alanine, taurine, trimethylglycine, and mixtures thereof.
